(19) United States
(12) Patent Application Publication
(10) Pub. No.: US 2013/0203721 A1
(43) Pub. Date: Aug. 8, 2013

(54) TASTE-MASKED POWDER FOR SUSPENSION COMPOSITIONS OF METHYLPREDNISOLONE

(71) Applicants: Pravin Meghrajji Bhutada, Pune (IN); Ashishi Ashokrao Deshmukh, Pune (IN); Prakash Bhagwat Gore, Pune (IN); Umesh Ganesh Badhe, Pune (IN); Sajeev Chandran, Pune (IN); Shirishkumar Kulkarni, Pune (IN)

(72) Inventors: Pravin Meghrajji Bhutada, Pune (IN); Ashishi Ashokrao Deshmukh, Pune (IN); Prakash Bhagwat Gore, Pune (IN); Umesh Ganesh Badhe, Pune (IN); Sajeev Chandran, Pune (IN); Shirishkumar Kulkarni, Pune (IN)

(73) Assignee: LUPIN LIMITED, Mumbai, Maharashtra (IN)

(21) Appl. No.: 13/580,513

(22) PCT Filed: Feb. 22, 2011
(86) PCT No.: PCT/IB11/00341

§ 371 Date: Oct. 31, 2012

(30) Foreign Application Priority Data
Feb. 22, 2010 (IN) 166/KOL/2010

(51) Int. Cl. A61K 9/14; A61K 31/573

(52) U.S. Cl. CPC A61K 9/14; A61K 31/573
USPC 514/179

(57) ABSTRACT

A dry taste masked powder composition comprising a steroid or its salts or derivatives and pharmaceutically acceptable excipients. The taste-masked powder may be used for suspension compositions suitable for use as a liquid suspension for children and elderly patients.

### FIELD OF THE INVENTION

The present invention relates to taste-masked powder for suspension compositions of methylprednisolone and its process of preparation.

### BACKGROUND OF THE INVENTION

Methylprednisolone, (6α,11β)-11,17,21-trihydroxy-6-methyl-pregna-1,4-diene-3,20 -dione, is a corticosteroid. It is a synthetic glucocorticoid drug similar to a natural hormone produced by adrenal glands. Methylprednisolone per se is sold in USA and Canada under the brand name Medrol® as 4, 8, 16 and 32 mg oral tablets. Methylprednisolone acetate and sodium succinate salts are available as injectable preparations. It relieves inflammation (swelling, heat, redness, and pain) and is used to treat certain forms of arthritis; skin, blood, kidney, eye, thyroid, and intestinal disorders (e.g., colitis); severe allergies; and asthma. Methylprednisolone is also used to treat certain types of cancer.

Methylprednisolone and some of its pharmaceutically acceptable salts were originally disclosed and claimed in U.S. Pat. No. 2,897,218 and GB 853,981 patents. These patents exemplify oral dosage forms including tablets, capsules, liquid suspensions or solutions of the drug. U.S. Pat. No. 4,874,613 patent discloses a pharmaceutical dosage unit (comprising a plurality of subdosage units disposed within a container) suitable for masking the unpleasant taste of orally administered methylprednisolone and which facilitates swallowing.

U.S. Pat. No. 5,874,418 patent describes sulfoalkyl ether cyclodextrin (SAE-CD) based formulations of methylprednisolone wherein a major portion of the therapeutic agent is not complexed with the SAE-CD and which is useful in modifying the bioavailability and/or rate of bioabsorption of therapeutic agents. SAE-CD based controlled release pharmaceutical formulations including multi-layered, osmotic pump, coated, and uncoated tablet, minitablet, pellet, micropellet, particle, powder, and granule dosage forms are disclosed in U.S. Pat. No. 6,046,177 patent. Aqueous, anti-inflammatory methylprednisolone compositions containing cyclodextrin in solution form suitable as nasal/oral/throat spray are disclosed in US 20060045850 A1 and US 20060120967 A1 patent applications.

Sublingual methylprednisolone formulations are disclosed in WO/1999/040898 patent application. US 20040265380 A1 application describes orodispersible effervescent tablets. US 20070059346 A1 application discloses orally-dissolvable, edible films of the drug. Mouth dissolving tablets as buccal dosage formulations of methylprednisolone are disclosed in US 20080317850 A1 application. US 20090263476 A1 application discloses a rapidly disintegrating buccal dosage form containing methylprednisolone, at least one non-effervescent base such as an alkali metal or alkaline earth metal oxide or hydroxide, and a disintegrant.

A wide variety of oral formulations have been suggested for methylprednisolone, but in practice the formulation most commonly used is a tablet, which may or may not be scored to assist in breaking it in half. Tablets, however, suffer from the disadvantage that they provide a fixed and inflexible dose of the steroid. While this may be quite acceptable or even desirable when treating adults, it represents a considerable problem to the physician when treating pediatrics and geriatrics patient group. Swallowing a tablet is difficult for children and elderly patients. In addition, in children, there is a considerable variation in body weight and thus very considerable variations in the amount of the steroid which is desired to be administered. Administering the right dose is particularly critical in case of steroids as an excessive dose can have growth retarding effects in growing children. Further in elderly patients, dose titration of steroids is necessary as the administered dose depends upon the severity of clinical manifestation of a disease or co-morbidity and accompanying concomitant medication being administered.

Liquid formulations are ideal candidates for pediatric and geriatric use as they are convenient to swallow and the dosage can be accurately controlled. U.S. Pat. No. 4,448,774 patent discloses aqueous pharmaceutical solution comprising methylprednisolone, preservative, chelating agent, and being substantially free of ethanol (particularly suitable for children). However, as methylprednisolone is very unpalatable, these types of solubilized preparations have the drawback of a lingering bitter after-taste and thus, poor patient compliance.

Another viable alternative to this limitation is to deliver the drug in the form of suspension. Ready-made suspensions, however, suffer from the disadvantage that on long storage they lack stability and with the passage of time they tend to settle down and even cake leading to inadequate or improper dosing.

There still exists a need for improved, stable and efficiently taste-masked oral compositions of methylprednisolone with good patient compliance and acceptance, especially for children and elderly patients.

There is also a need for a simple efficient process for making such a taste-masked powder for suspension compositions having easy reconstitution, better in-use stability and prolonged shelf life.

### SUMMARY OF THE INVENTION

One embodiment provides dry taste-masked powder for suspension compositions suitable for use as a liquid suspension for children or elderly patients. The compositions include methylprednisolone or its salt/derivative and pharmaceutically acceptable excipients selected from the group consisting of suspending agents, viscosity enhancers, coating agents, preservatives, flavouring agents, sweeteners, opacifiers, lubricants, glidants, wetting agents, surfactants, buffering agents, and diluents.

Another embodiment provides the enhanced taste-masking capability of compositions by granulating, coating, complexing with ion-exchange resin, forming inclusion complex of the drug with cyclodextrin or forming solid-dispersion before admixing the drug with other additives or excipients.

Further, embodiment provides a taste-masked composition of methylprednisolone, wherein the amount of methylprednisolone varies from about 0.01-50% by weight of the dry taste-masked powder for suspension composition. Upon reconstitution, the amount of drug in the oral suspensions ranges from 2 to 40 mg/5 mL and preferably from 4 to 24 mg/5 mL.

Still further embodiment provides a method of preparing a dry methylprednisolone powder for suspension composition, which is suitable for suspension in water and/or water miscible suitable solvents to form an orally administerable product which comprises admixing, at ambient temperature and humidity conditions, dried taste-masked methylpredniosolone granules with substantially dry pharmaceutically acceptable excipients selected from the group consisting of suspending agents, viscosity enhancers, coating agents, preservatives, flavouring agents, sweeteners, opacifiers, lubricants, glidants, wetting agents, surfactants, buffering agents, and diluents to form a dry admixture, and transferring the dry admixture to a sealable storage container.

Still further embodiment provides process for masking the bitter, lingering taste of methylpredniosolone. The process includes, but is not limited to (i) simple admixture of drug and taste-masking agent (e.g., sweetener, flavour, etc) followed by granulation & drying, and optionally coating; (ii) loading non-pareil beads with drug followed by coating with a polymer, which dissolves/disintegrates in acidic pH of stomach, but is resistant to salivary pH; (iii) mixing drug with thermodiffusible taste-masking substance; (iv) complexing the drug with ion-exchange resin; (v) forming inclusion complex of drug with cyclodextrin; and (vi) forming solid-dispersion of the drug.

### DETAILED DESCRIPTION OF THE INVENTION

In view of the description presented in the background, it would be advantageous to develop an oral taste-masked powder for suspension compositions suitable for use as a liquid suspension for children and elderly patients.

Further, it would be advantageous to provide a process for the preparation of such a suspension that is convenient, economical, and requires simple machinery.

It has now been unexpectably found that the unpleasant taste and stability problem may be addressed by employing methylprednisolone in taste-masked powder for suspension compositions. The external vehicle is pleasantly flavoured and the insoluble drug remains suspended, does not come in direct contact with the tongue and thus, does not impart bitter taste to the preparation. To further enhance the organoleptic properties, other simple and cost effective techniques may be employed to taste-mask the bitter drug before suspending. Since the preparation is reconstituted just prior to usage, its physical & chemical stability is enhanced and caking problem & uneven dosing is avoided. The suspension formed is particularly convenient for pediatric and geriatric patients.

“Methylprednisolone”, as used includes methylprednisolone and its salts, esters or, derivatives. The compositions include a mixture of methylprednisolone and pharmaceutically acceptable excipients selected from the group consisting of suspending agents, viscosity enhancers, coating agents, preservatives, flavouring agents, sweeteners, opacifiers, lubricants, glidants, wetting agents, surfactants, buffering agents, and diluents. Methylprednisolone may be present at up to about 50% by weight of the dry powder for suspension composition. Upon reconstitution, each 5 mL of oral suspension may contain up to 40 mg and preferably 4 to 24 mg of methylprednisolone.

“Dry powder”, as used herein, includes any composition which is dry and flowable such as, for example, granules, flakes, spheroids and other forms which can be readily prepared and when added to an ingestible liquid and mixed, give the desired liquid suspension.

The term “suspending agent” or “viscosity enhancer”, as used herein, refers to an agent or a mixture of agents that increases the thickness of a liquid thereby making it slow to flow. For example, in a suspension a viscosity enhancer will help to keep the active ingredient suspended to allow accurate dosing. They may be selected from one or more cellulosic derivatives, gums, polysaccharides, alginates, acrylic acid copoymers, polyvinyl pyrrolidone, or combinations thereof Representative suspending agents or viscosity enhancers include, but are not limited to, acacia, alginic acid, bentonite, carbomer, carboxymethylcellulose calcium, carrageenan, colloidal silicon dioxide, dextrin, gelatin, guar gum, hydroxyl ethyl cellulose, hydroxyethyl propylcellulose, hydroxyl propyl cellulose (HPC), hydroxypropyl methylcellulose, methylcellulose, maltodextrin, microcrystalline cellulose (MCC), polydextrose, polyvinyl alcohol, povidone, propylene glycol alginate, sodium alginate, sodium carboxymethylcellulose, starch, tragacanth, xantham gum, and mixtures thereof Preferred suspending agents or viscosity enhancers are Avicel® CL 611 and mixture of xanthan gum & HPC (Klucel® LF).

The term “preservative”, as used herein, refers to an agent or mixture of agents that is used to protect a composition against microbes (e.g., yeast, mold; bacteria, etc). Representative preservatives include, but are not limited to, benzalkonium chloride, benzethonium chloride, benzoic acid, benzyl alcohol, bronopol, butyl paraben, chlorobutanol, chlorocresol, chlorhexidine, cresol, ethylenediaminetetraacetic acid, ethyl paraben, imidurea, methyl paraben, phenol, phenylmercuric salts, potassium sorbate, propylene glycol, propyl paraben, sodium benzoate, sodium citrate, sodium propionate, sorbic acid, thiomerosol, and mixtures thereof A preferred preservative is sodium benzoate or mixture of parabens, which impart synergistic preservative action.

The term “flavouring agent”, as used herein, refers to an agent or a mixture of agents that is natural or artificial in origin and adds flavour to a mixture. Representative flavouring agents include, but are not limited to, banana, black currant, caramel, cherry, chocolate, cream, grenadine, mint, raspberry, strawberry, tutti frutti flavours, and mixtures thereof. To enhance organoleptic properties, combination of flavours may be used. For example, strawberry and banana flavours may be used together.

The term “sweetener”, as used herein, refers to both bulk (caloric) and intense (non-caloric) sweeteners, which impart sweet taste to the preparation. Examples of bulk sweeteners are dextrose, fructose, glucose, hydrogenated glucose syrup, isomalt, maltitol, maltose, mannitol, sorbitol, sucrose, xylitol, and mixtures thereof. Examples of intense sweeteners are acesulfame, alitame, aspartame, cyclamate, dihydrochalcone sweetener, monellin, neohesperidin, neotame, saccharin, stevioside, sucralose, the pharmaceutically acceptable salts thereof such as sodium or calcium saccharin, acesulfame potassium or sodium cyclamate, and mixtures thereof. Sweetener type, combination and proportion may be varied in various compositions. For example, acesulfame potassium at a proportion of 0-0.9% by weight and sucrose at a proportion of 0-90% by weight may be used alone or in combination.

The term “opacifier”, as used herein refers to an agent or a mixture of agents which when added to a preparation make the ensuing system opaque. Representative opacifier agents include, but are not limited to, pharmaceutically acceptable metal oxides, especially titanium dioxide.

The term “lubricant”, as used herein refers to an agent or a mixture of agents that lessens or prevents friction. Representative lubricants include, but are not limited to, calcium stearate, colloidal silicon dioxide, corn starch, hydrogenated vegetable oil, magnesium oxide, magnesium stearate, sodium lauryl stearate, sodium stearyl fumarate, stearic acid, talc, zinc stearate, and mixtures thereof. A preferred lubricant is sodium stearyl fumarate.

The term “glidant”, as used herein, refers to an agent or a mixture of agents that facilitates the flow of powders in the manufacturing process. Representative glidants include, but are not limited to, calcium stearate, colloidal silicon dioxide, fumed silica, magnesium stearate, magnesium trisilicate, powdered cellulose, starch, talc, tribasic calcium phosphate, and mixtures thereof. A preferred glidant is fumed silica (Aerosil® 200).

The term “wetting agent” or “surfactant”, as used herein, refers to a surface active agent or a mixture of agents that lower the interfacial tension between a solid & a liquid or two liquids. It may be natural or synthetic in origin. Further, it may be non-ionic, anionic, cationic or amphoteric in nature. Representative examples include, but are not limited to, acacia, poloxamers, polysorbates such as Tween® 40, 60 & 80, sodium lauryl sulphate, sodium monolaurate (Span® 20), tragacanth, and mixtures thereof

The term “buffering agent”, as used herein, refers to an agent or a mixture of agents that can maintain the original acidity or basicity of a composition. Representative buffering agents include, but are not limited to, citric acid, disodium hydrogen phosphate, lactic acid, monosodium citrate, phosphoric acid, potassium citrate, sodium citrate, sodium hydrogen carbonate, sodium hydroxide, sodium phosphate, succinic acid, tartaric acid, and mixtures thereof The amount of buffering agent used is dependent on (a) the desired pH; and (b) the amount of methylprednisolone per mL of the suspension.

The term “diluent”, as used herein, refers to an agent or mixture of agents that when added to a formulation makes that formulation thinner or less concentrated or acts as filler and may also improve manufacturability. Diluents can also serve other functions. For example, a diluent can also act as a sweetener and/or a suspending agent. Representative diluents include, but are not limited to, dextrose, fructose, lactose, maltitol, microcrystalline cellulose, sorbitol, starch, sucrose, xylitol, and mixtures thereof Preferred diluents are sucrose and microcrystalline cellulose.

The term “non-pareil sphere” or “pellet”, as used herein, refers to an agent or a mixture of agents that otherwise is inert, but acts as an excellent active pharmaceutical ingredient carrier. Representative examples include, but are not limited to, sucrose, microcrystalline cellulose and starch.

The term “taste-masking pH-sensitive polymer”, as used herein, refers to a polymer or a mixture of polymers, which disintegrate or dissolve at acidic pH in the stomach but are resistant to near neutral pH in the mouth. Representative examples include, but are not limited to, cationic polymers synthesized from dimethylaminoethyl methacrylate and neutral methacrylic acid esters alone or in combination with small quantities of retardant polymers like, ethyl cellulose, Eudragit®, hydroxyl propyl cellulose and hydroxypropyl methyl cellulose. These polymers may also be used as coating agents. Preferred taste-masking pH-sensitive polymers are Eudragit® E 100 and Eudragit® E PO.

The term “plasticizer”, as used herein, refers to an agent or a mixture of agents that increases the plasticity or fluidity of the material to which it is added and imparts softness and flexibility to the final product, which may be a coat. Representative examples include, but are not limited to, dibutyl sebacate, glycerol, propylene glycol, triethyl citrate, and mixtures thereof.

The term “polyethylene glycol”, as used herein, refers to an agent or a mixture of agents, belonging to a family of water-soluble linear polymers, which melt at not more than 60° C. and solidify at room temperature. Representative examples include, but are not limited to various viscosity grades of polyethylene glycol (PEG) like, PEG 1500, PEG 3350, PEG 6000, and mixtures thereof.

The term “antioxidant”, as used herein, refers to an agent or a mixture of agents, which are capable of slowing or preventing the oxidation of other molecules or substances. Representative examples include, but are not limited to, ascorbic acid, butylated hydroxy anisole, butylated hydroxy toluene, gallic acid, maleic acid, propyl gallate, sodium bisulphate, sodium metabisulphite, tocopherols, and mixtures thereof.

The term “solvent”, as used herein, refers to an agent or a mixture of agents in the form of a liquid, which is used for dissolving another solid or liquid, or as a dispersing or granulating media. Solvent employed may be aqueous, hydroalcoholic or organic in nature with varying polarity. Preferred alcohols employed in hydroalcoholic solvent system include, but are not limited to, ethyl alcohol and isopropyl alcohol alone or in combination. Representative examples of solvent employed include, but are not limited to, acetone, dichloromethane, ethyl alcohol, isopropyl alcohol, water, and mixtures thereof.

The term “ion-exchange resin”, as used herein, refers to an agent or a mixture of agents, which can exchange their mobile ions of equal charge with the surrounding medium. Being high molecular weight water insoluble polyelectrolytes, the resins are not absorbed by the body and are therefore inert. For optimal taste-masking, drug to ion-exchange resin ratio may be varied from 1:0.1 to 0.1:1. Representative examples of ion-exchange resins include, but are not limited to, Amberlite® CG 50, Amberlite® IRP-64, Amberlite® IRP-69, Indion® 204, Indion® 214, Indion® 234, Indion® Indion CRP 244, Indion® CRP 254, and mixtures thereof.

The term “cyclodextrin”, as used herein, refers to an agent or a mixture of agents, capable of forming stable inclusion complex and masking the bitter taste of the drug by either decreasing its oral solubility on ingestion or decreasing the amount of drug particles exposed to taste buds thereby reducing the perception of bitter taste. For optimal taste-masking, drug to cyclodextrin ratio may be varied from 1:0.1 to 0.1:1. Representative examples of cyclodextrin include, but are not limited to, alpha cyclodextrin, gamma cyclodextrin, beta cyclodextrin, cyclodextrin derivative, and mixtures thereof. A preferred cyclodextrin is beta cyclodextrin.

The term “thermodiffusible substance”, as used herein, refers to an agent or a mixture of agents, which are liquid at elevated temperatures and solid at room temperature. For optimal taste-masking, drug to thermodiffusible substance ratio may be varied from 1:0.1 to 0.1:1. Representative examples of thermodiffusible substances include, but are not limited to, beeswax, candelilla wax, carnauba wax, glyceryl behenate, microcrystalline wax, oricury, ozokerite, paraffin wax, polyethylene glycol (PEG) wax, PEG 1500, PEG 3350, PEG 6000, hydrogenated vegetable oil, shellac, and mixtures thereof

The term “triglyceride”, as used herein, refers to small chained, medium chained or long chained triglyceride, which is an ester of fatty acids and glycerol. Suitable triglycerides for use may be natural, semi-synthetic or synthetic in origin. Representative examples of triglycerides include, but are not limited to, Acomed®, Captex®, Mazol®, Miglyol®, Myritol®, Neobee®, Sefsol®; and naturally occurring or partially/fully hydrogenated plant, vegetable & fish oils such as castor oil, coconut oil, olive oil, palm oil, peanut oil, sesame oil, shark oil, soybean oil, sunflower oil, etc, and mixtures thereof. Preferred triglycerides are Captex® 350 and Miglyol® 812.

The term “solid-dispersion”, as used herein, refers to a product formed when the drug is made to interact physically or chemically with one or more hydrophilic or hydrophobic substances. The product formed has improved organoleptic properties and optionally, altered solubility and/or altered physicochemical properties. Representative examples of hydrophilic/hydrophobic substances capable of forming a solid-dispersion include, but are not limited to, ethyl cellulose, Eudragit®, mannitol, poloxamer, polyethylene glycol, sorbitol, sucrose, urea, and polymers, like carbopol, copovidone, polyvinyl alcohol, polyvinyl pyrrolidone, hydroxypropyl methyl cellulose, and mixtures thereof

Pharmaceutical compositions as described herein are presented as dry powder suspension composition for constitution with an ingestible liquid such as water before use. The dry powder for suspension composition can be packed in suitable containers to provide multi-dose liquid suspension.

The dry powder after reconstitution with water is set to provide a liquid suspension containing 4 to 24 mg of methylprednisolone per 5 mL of liquid suspension. The dry powder for suspension composition is stable on storage and when constituted with an ingestible liquid for administration, the corresponding liquid suspension is stable physically and chemically for the duration in which the therapy is required.

The dry powder for suspension composition may be prepared by the procedure that includes, but is not limited to, dry blending of methylprednisolone as such or taste-masked drug and pharmaceutically acceptable excipients selected from the group consisting of suspending agents, viscosity enhancers, coating agents, preservatives, flavouring agents, sweeteners, opacifiers, lubricants, glidants, wetting agents, surfactants, buffering agents, and diluents. These are then filled into a bottle or a suitable container in an amount suited to a dosage regimen. A suitable ingestible liquid, particularly water, is added in an amount sufficient to provide methylprednisolne in desired dosage strength.

Embodiments include one or more of the following features. For example, the composition may exhibit enhanced taste-masking capability by granulating methylprednisolone with sweeteners & flavours, and optionally coating with coating agents; coating drug loaded non-pareil beads with taste-masking polymers; complexing methylprednisolne with ion-exchange resin; forming inclusion complex of the drug with cyclodextrin; or forming solid-dispersion of the drug before admixing with other additives or excipients at ambient temperature and humidity conditions and transferring into sealable storage container.

The following examples illustrate various aspects of the invention; but are not intended to be limiting the scope of the invention.

### EXAMPLES

### Example 1

| S. No. | Ingredients | % w/w* |
|---|---|---|
| 1 | Methylprednisolone | 0.01-20 |
| 2 | Sucrose | 0-90 |
| 3 | Sucralose | 0-10 |
| 4 | Avicel ® CL 611 | 0-10 |
| 5 | Hydroxy propyl cellulose | 0-10 |
| 6 | Xanthan gum | 0-10 |
| 7 | Titanitim dioxide | 0-5 |
| 8 | Sodium benzoate | 0.05-1 |
| 9 | Butylated hydroxy anisole | 0-5 |
| 10 | Cherry and banana flavour mixture | 0-2 |
| 11 | Aerosil ® 200 | 0-2 |
| 12 | Sodium stearyl fumarate | 0.2-1 |
| 13 | Sodium citrate | 0-5 |
| 14 | Citric acid | 0-1.5 |
| 15 | Isopropyl alcohol and water mixture | q.s. |

*On dry weight basis of final product

Brief Manufacturing Procedure:
1) Granulate methylprednisolone, sucrose & sucralose with isopropyl alcohol & water mixture and dry.
2) Sift the dried granules using SS sieve #30 mesh.
3) Blend the sifted granules with Avicel® CL 611, hydroxy, propyl cellulose, xanthan gum, titanium dioxide, flavour mixture, Aerosil® 200, sodium benzoate, butylated hydroxy anisole, sodium citrate, citric acid and sodium stearyl fumarate.
4) Fill in dry powder for suspension composition into final container.

### Example 2

Step 1 - Methylprednisolone loading on non-pareil spheres and coating

| S. No. | Ingredients | % w/w* |
|---|---|---|
| 1 | Non-pareil spheres^{@} | 20-40 |
| 2 | Methylprednisolone | 5-50 |
| 3 | Ethyl cellulose 10 cps | 0-5 |
| 4 | Hydroxy propyl cellulose | 0-5 |
| 5 | Dibutyl sebacate | 0-1 |
| 6 | Eudragit ® E 100 | 5-20 |
| 7 | Talc | 1-2 |
| 8 | Acetone and dichloromethane mixture | q.s. |
| 9 | Acetone and isopropyl alcohol mixture | q.s. |

*On dry weight basis of drug loaded non-pareil sphere coated with Eudragit
^{@}Particle size distribution of unloaded spheres range from about 250 to 600 μm or (sieve # 45/60 to sieve # 35/40).

Step 2 - Preparation of suspension blend

| S. No. | Ingredients | % w/w* |
|---|---|---|
| 1 | Coated methylprednisolone loaded | 2-50 |
| | non-pareil spheres | |
| 2 | Sucrose | 0-90 |
| 3 | Hydroxy propyl cellulose | 0-5 |
| 4 | Sucralose | 0-10 |
| 5 | Xanthan gum | 0-5 |
| 6 | Titanium dioxide | 0-2 |
| 7 | Strawberry flavour | 0-2 |
| 8 | Caramel flavour | 0-2 |
| 9 | Sodium citrate | 0-5 |
| 10 | Citric acid | 0-1.5 |
| 11 | Aerosil ® 200 | 0-1 |
| 12 | Magnesium stearate | 0.2-1 |

*On dry weight basis of final product

Brief Manufacturing Procedure:
1) Dissolve methylprednisolone, ethyl cellulose, and hydroxy propyl cellulose in acetone & dichloromethane mixture.
2) Load the non-pareil beads with the solution of step 1 in fluidized bed dryer. Dry the loaded beads.
3) Prepare solution of Eudragit® E 100 in acetone & isopropyl alcohol mixture and coat the drug loaded beads in fluidized bed dryer.
4) Blend the resultant granules with suspension blend of step 2 in rapid mixer.
5) Fill in dry powder for suspension composition into final container.

### Example 3

| S. No | Ingredients | % w/w* |
|---|---|---|
| 1 | Methylprednisolone | 0.01-20 |
| 2 | Polyethylene glycol | 0.5-18.5 |
| 3 | Sodium metabisulphite | 0.03-0.1 |
| 4 | Sucralose | 0.5-5 |
| 5 | Maltodextrin | 0-15.9 |
| 6 | Avicel ® CL 611 | 0-10 |
| 7 | Xanthan gum | 0-10 |
| 8 | Sucrose | 2.6-31.7 |
| 9 | Strawberry and banana flavour mixture | 0.2-0.5 |
| 10 | Acetone | q.s. |

*On dry weight basis of final product

Brief Manufacturing Procedure:
1) Melt polyethylene glycol (below 60° C.) and add sodium metabisulphite to the molten mass under continuous stirring. Allow the molten mass to solidify. Mill the solidified mass using SS sieve #30 mesh.
2) Pass drug, sucralose, maltodextrin, Avicel® CL 611 and sucrose through SS sieve #30 mesh and mix them geometrically in rapid mixer granulator.
3) Add the milled and sieved mass obtained in step 1 to the blend obtained in step 2 in rapid mixer granulator and mix to obtain homogeneous blend.
4) Dissolve sodium benzoate in acetone and use it for granulating the blend obtained in step 3.
5) Dry the wet granules of step 4 and sift the dried granules through. SS sieve #20 mesh.
6) Lubricate the granules of Step 5 with flavour mixture.
7) Fill in dry powder for suspension composition into final container.

### Example 4

Step 1 - Formation of drug-resin complex

| S. No. | Ingredients | % w/w* |
|---|---|---|
| 1 | Methylprednisolone sodium succinate | 25-67 |
| 2 | Amberlite ® IRP-64 | 33-75 |
| 3 | Ethyl alcohol and water mixture | q.s. |
| 4 | Isopropyl alcohol | q.s. |

*On dry weight basis of drug-resin complex

Step 2 - Preparation of suspension blend

| S. No. | Ingredients | % w/w* |
|---|---|---|
| 1 | Methylprednisolone-resin complex | 1-20 |
| 2 | Sucrose | 0-90 |
| 3 | Sodium carboxymethylcellulose | 0-10 |
| 4 | Hydroxy propyl cellulose | 0-5 |
| 5 | Xanthan gum | 0-5 |
| 6 | Titanium dioxide | 0-2 |
| 7 | Strawberry flavour | 0-2 |
| 8 | Caramel flavour | 0-2 |
| 9 | Aerosil ® 200 | 0-1 |
| 10 | Calcium stearate | 0-2 |

*On dry weight basis of final product

Brief Manufacturing Procedure:
1) Dissolve methylprednisolone sodium succinate in ethyl alcohol & water mixture and add known weight of ion-exchange resin to the solution under stirring to obtain uniform slurry.
2) Adjust the pH of the slurry using acidic or alkaline buffering agent.
3) Continue stirring until drug-resin complexation equilibrium is achieved.
4) Wash obtained resinates with minimum amount of isopropyl alcohol to remove uncomplexed drug.
5) Dry the complexes overnight in a hot air oven at 40° C. and then store in tightly closed dessicator.
6) Mix the above obtained dried resinates with the suspension blend of step 2.
7) Fill in dry powder for suspension composition into final container.

### Example 5

Step 1 - Formation of inclusion complex

| S. No. | Ingredients. | % w/w* |
|---|---|---|
| 1 | Methylprednisolone | 1-50 |
| 2 | Cyclodextrin | 5-90 |
| 3 | Polyvinyl pyrrolidone | 0-10 |
| 4 | Acetone | q.s. |

*On dry weight basis of drug-cyclodextrin complex

Step 2 - Preparation of suspension blend

| S. No. | Ingredients | % w/w* |
|---|---|---|
| 1 | Methylprednisolone-cyclodextrin complex | 2-10 |
| 2 | Sucrose | 0-90 |
| 3 | Hydroxy propyl cellulose | 0.09-0.11 |
| 4 | Sucralose | 0-10 |
| 5 | Xanthan gum | 0.09-0.11 |
| 6 | Titanium dioxide | 0.7-0.8 |
| 7 | Cherry and banana flavour mixture | 0.2-2 |
| 8 | Aerosil ® 200 | 0.5-2 |
| 9 | Sodium stearyl fumarate | 0.2-1 |

*On dry weight basis of final product

Brief Manufacturing Procedure:
1) Granulate methylprednisolone, cyclodextrin, and polyvinyl pyrrolidone with acetone and dry.
2) Sift the dried granule using SS sieve #30 mesh.
3) Mix the sifted granules with sucrose, sucralose, hydroxy propyl cellulose, xanthan gum, titanium dioxide, flavour mixture, and Aerosil® 200.
4) Lubricate the final blend with sodium stearyl fumarate.
5) Fill in dry powder for suspension composition into final container.

### Example 6

| S. No. | Ingredients | % w/w* |
|---|---|---|
| 1 | Methylprednisolone | 0.01-50 |
| 2 | Polyethylene glycol | 0-90 |
| 3 | Poloxamer | 0-25 |
| 4 | Carrageenan | 0-5 |
| 5 | Hydroxy propyl cellulose | 0-10 |
| 6 | Xanthan gum | 0-10 |
| 7 | Titanium dioxide | 0-10 |
| 8 | Sucralose | 0-10 |
| 9 | Banana flavour | 0-5 |
| 10 | Aerosil ® 200 | 0-2 |
| 11 | Magnesium stearate | 0.2-1 |

*On dry weight basis of final product

Brief Manufacturing Procedure:
1) Melt polyethylene glycol.
2) Add, to the above molten mass, drug & poloxamer and stir.
3) Congeal the above mixture using spray congealing technique.
4) Mill and sift the above material using SS sieve #30 mesh.
5) Blend the sifted material with carrageenan, sucralose, hydroxyl propyl cellulose, xanthan gum, titanium dioxide, flavour, and Aerose 200.
6) Lubricate the final blend with magnesium stearate.
7) Fill in dry powder for suspension composition into final container.

### Example 7

Step 1 - Formation of drug-lipid granulate

| S. No. | Ingredients | % w/w* |
|---|---|---|
| 1 | Methylprednisolone | 0.01-10 |
| 2 | Miglyol ® 812 | 1-20 |
| 3 | Poloxamer | 0-5 |
| 4 | Sucrose Pellets | 0-5 |
| 5 | Acetone | q.s. |

*On dry weight basis of drug-lipid granulate

Step 2 - Preparation of suspension blend

| S. No. | Ingredients | % w/w* |
|---|---|---|
| 1 | Drug-lipid granulate | 2-10 |
| 2 | Xanthan gum | 2-20 |
| 3 | Microcrystalline cellulose | 0-20 |
| 4 | Sucralose | 0-10 |
| 5 | Sucrose | 0-90 |
| 6 | Strawberry and banana flavour mixture | 0-5 |
| 7 | Sodium stearyl fumarate | 0.5-3 |

*On dry weight basis of final product

Brief Manufacturing Procedure:
1) Dissolve or disperse methylprednisolone and Miglyol 812 along with poloxamer in sufficient quantity of acetone. Mix it for sufficient time.
2) Adsorb the above dispersion on sucrose pellets.
3) Dry the drug-lipid granulates and sift it through SS sieve #30 mesh.
4) Blend the dried drug-lipid granules with xanthan gum, microcrystalline cellulose, sucrose, sucralose and flavour mixutre.
5) Lubricate the final blend with sodium stearyl fumarate.
6) Fill in dry powder for suspension composition into final container.

### Example 8

Step 1 - Formation of solid-dispersion

| S. No. | Ingredients | % w/w* |
|---|---|---|
| 1 | Methylprednisolone | 0.1-50 |
| 2 | Kollidon ® VA 64 | 5-80 |
| 3 | Isopropyl alcohol and | q.s. |
| | dichloromethane mixture | |

*On dry weight basis of drug-lipid granulate

Step 2 - Preparation of suspension blend

| S. No. | Ingredients | % w/w* |
|---|---|---|
| 1 | Drug-Kollidon solid-dispersion | 1-10 |
| 2 | Xanthan gum | 0-10 |
| 3 | Microcrystalline cellulose | 0-10 |
| 4 | Sucrose | 0-90 |
| 5 | Sucralose | 0-10 |
| 6 | Strawberry and banana flavour mixture | 0-5 |
| 7 | Sodium stearyl fumarate | 0.5-3 |

*On dry weight basis of final product

Brief Manufacturing Procedure:
1) Dissolve and mix methylprednisolone and Kollidon® VA 64 in sufficient quantity of isopropyl alcohol & dichloromethane mixture. Dry the solution in vacuum try drier and sift it through SS sieve #30 mesh.
2) Blend the dried drug-Kollidon solid-dispersion with xanthan gum, microcrystalline cellulose, sucrose, sucralose, and flavour mixutre.
3) Lubricate the final blend with sodium stearyl fumarate.
4) Fill in dry powder for suspension composition into final container.

### Example 9

| S. No. | Ingredients | % w/w* |
|---|---|---|
| 1 | Methylprednisolone | 0.01-10 |
| 2 | Eudragit ® E PO | 0-25 |
| 3 | Polyethylene glycol | 0-90 |
| 4 | Sucrose | 0-90 |
| 5 | Avicel ® CL 611 | 0-10 |
| 6 | Hydroxy propyl cellulose | 0-10 |
| 7 | Carrageenan | 0-10 |
| 8 | Titanium dioxide | 0-5 |
| 9 | Sodium benzoate | 0.05-1 |
| 10 | Butylated hydroxy anisole | 0-5 |
| 11 | Cherry and banana flavour mixture | 0-2 |
| 12 | Acesulfame | 0-5 |
| 13 | Aerosil ® 200 | 0-2 |
| 14 | Sodium stearyl fumarate | 0.2-1 |
| 15 | Sodium citrate | 0-5 |
| 16 | Citric acid | 0-1.5 |
| 17 | Acetone, ethyl alcohol and | q.s. |
| | isopropyl alcohol mixture | |

*On dry weight basis of final product

Brief Manufacturing Procedure:
1) Dissolve methylprednisolone & part of flavour mixture, and optionally polyethylene glycol, in acetone, ethyl alcohol & isopropyl alcohol mixture.
2) Mix Avicel® CL 611 with sucrose in a rapid mixer granulator and granulate the mixture with the solution obtained above.
3) Dry and sift the granules using SS sieve #30 mesh. Optionally, coat the obtained granules with Eudragit® E PO dispersion or solution.
4) Blend the sifted granules with hydroxyl propyl cellulose, sodium benzoate, butylated hydroxy anisole, acesulfame, carrageenan, titanium dioxide, flavour mixture, Aerosil® 200, sodium citrate, citric acid, and sodium stearyl fumarate.
5) Fill in dry powder for suspension composition into final container.

1. A dry taste masked powder composition comprising a steroid or its salts or derivatives and pharmaceutically acceptable excipients.

2. The composition of claim 1 comprising one or more pharmaceutically acceptable excipients selected from the group consisting of suspending agent, viscosity enhancer, coating agents, preservatives, flavouring agents, sweeteners, opacifiers, lubricants, glidants, wetting agents, surfactants, resins, buffering agents, complexing agents and diluents.

3. The composition of claim 1 wherein the steroid is methylprednisolone.

4. The composition of claim 1 wherein the composition is in the form of a liquid suspension upon reconstitution.

5. The composition of claim 3 comprising the steroid in the range of about 0.01-50% by weight of the dry taste masked powder.

6. The composition according to claim comprising 2 to 40 mg/5 ml of steroid.

7. The dry taste masked powder composition according to claim 14 comprising granules, drug lipid granulates, non pareil spheres, spheroids, or flakes.

8. The composition according to claim 1 wherein the process for preparation of dry taste masked powder is selected from solid dispersion, granulation, complexation, coating, or solvent evaporation reconstitution.

9-17. (canceled)
